(19) United States
(12) Patent Application Publication
(10) Pub. No.: US 2013/0123609 A1
(43) Pub. Date: May 16, 2013

(54) Flexible Coil for MR Experiments on Small Animals

(71) Applicants: Florian Odoj, Rimpar (DE); Titus Lanz, Rimpar (DE)

(72) Inventors: Florian Odoj, Rimpar (DE); Titus Lanz, Rimpar (DE)

(73) Assignee: RAPID Biomedical GmbH, Rimpar (DE)

(21) Appl. No.: 13/464,035

(22) Filed: May 4, 2012

Related U.S. Application Data
(60) Provisional application No. 61/483,281, filed on May 6, 2011.
(60) Provisional application No. 61/483,256, filed on May 6, 2011.

(51) Int. Cl. A61B 5/055; A61B 5/00; A61B 6/00

(52) U.S. Cl. CPC A61B 5/055; A61B 6/44; A61B 5/0035
USPC 600/411; 600/422; 600/425

(57) ABSTRACT

A flexible coil device for supporting imaging experiments on subjects, such as small animals. The device has a rigid part where electronics are accommodated and a flexible loop acting as a receiving coil that picks up the imaging signal. The flexible loop allows its shape to be adapted to the particular shape of the imaged subject at the point of placement. This provides a better filling factor and hence better SNR of the region of interest (ROI). This has the advantage that various imaging applications pose less of a compromise than with rigid coils. The flexible loop can be positioned right against the region of interest (ROI) and so maximises the filling factor and the image SNR.

### CROSS-REFERENCES TO RELATED APPLICATIONS

This application claims the benefit of U.S. provisional application Ser. No. 61/483,256 filed on May 6, 2011, incorporated herein by reference. This application also claims the benefit of U.S. provisional application Ser. No. 61/483,281 filed on May 6, 2011, and also incorporated herein by reference.

### BACKGROUND OF THE INVENTION

This application relates generally to a device for receiving an imaging signal from an animal during an imaging operation.

More specifically, this application relates to an apparatus and method for flexibly imaging various parts of an anesthetized subject such as an animal or rodent (rats or mice or other) in real-time in a non-destructive manner.

Rodents and other laboratory animals are often used for testing purposes. Such testing may involve the need to scan the animal using a scanning device, such as a SPECT, PET, CT, CAT, X-Ray, NMR/MR, or other imaging device, to provide real time and/or photographic images of the animal, which may be done in a non-destructive manner. It is often desirable to image various parts of an anesthetized animal. Desirable is an imaging device that offers flexibility in where it can be placed and how it can be utilized.

Investigations on small animals usually pose problems as the structures to be investigated are very small and often have a complex shape. For optimum performance one should have dedicated small animal coils for the particular application and size of animal. However, there are practical limitations to the coil size and shape and often it is simply not viable to have various coils for different applications or animal sizes.

Existing coils can be grouped in whole body volume coils, general small surface coils or dedicated brain surface coils or heart surface coils. All these coils have so far been produced in a rigid housing. The whole body volume coil is designed so that the whole animal fits inside the coil and can be imaged. This has the advantage that the complete animal can be investigated in one scan. However the achieved local signal-to-noise ratio (SNR) is much lower compared to a surface coil. The housing of the dedicated surface coils is matched to the particular application and usually provides a good match to the shape of the imaged object. The “general purpose” small surface coil is not matched to a particular application and is commonly housed in a cuboid or a curved box.

With state-of-the-art small surface coils it is difficult to position small animals in a way that the region of interest (ROI) is well covered by the coil's field of view. The positioning is arranged so that the investigated animal is shaped by the housing of the coil which often is not a physiological posture.

Desirable is a device for providing the proper positioning of the animal in a physiological posture using coils with sufficient SNR and matched to the particular application.

### SUMMARY OF THE INVENTION

Provided as an example embodiment is a small flexible coil device for imaging (e.g., Magnetic Resonance Imaging (MRI)) experiments on subjects such as small animals. The device has a rigid part where the electronics are accommodated and a flexible loop acting as a receiving coil that picks up the MR signal. The flexible loop allows its shape to be adapted and adjusted to the particular shape of the imaged subject. This provides a better filling factor and hence better SNR of the region of interest (ROI). This has the advantage that various imaging applications pose less of a compromise than with rigid coils. The flexible loop can be positioned right against the region of interest (ROI) and so maximises the filling factor and the image SNR.

Also provided is a device for receiving an imaging signal, comprising; a coil loop for receiving the imaging signal; processing electronics for processing a signal received by the coil loop; a base portion for enclosing the processing electronics; a flexible portion for enclosing the receiving coil, wherein the flexible portion is adapted for conforming to any one of a number of different locations on the body of a subject (human or animal) for receiving the imaging signal from a desired region of interest of the subject.

Further provided is a device for receiving an imaging signal, comprising; a receiving coil for receiving the imaging signal; processing electronics for amplifying a signal received by the receiving coil into a signal for output by the device; a rigid base for mounting the processing electronics thereon; a base portion for enclosing the processing electronics and rigid base; and a flexible portion for enclosing the receiving coil, wherein the flexible portion is adapted for conforming to any one of a number of different locations on the body of an animal for receiving the imaging signal from a desired region of interest of the animal.

Also provided is a device for receiving an imaging signal, comprising; a receiving coil for receiving the imaging signal; processing electronics for processing a signal received by the receiving coil; a rigid base for mounting the processing electronics thereon; an individually formed top part made of a flexible material; and an individually formed bottom part made of a flexible material.

For the above device, the top part and the bottom part are arranged and assembled together in a manner forming a base portion for enclosing the processing electronics and the rigid base. Also for the above device, the top part and the bottom part are also arranged together in a manner to form a flexible portion for enclosing the receiving coil. In addition, for the above device, the flexible portion is adapted for conforming to any one of a number of different locations on the body of an animal held in place for receiving a signal from a desired region of interest of the animal.

### BRIEF DESCRIPTION OF THE DRAWINGS

The features and advantages of the example embodiments described herein will become apparent to those skilled in the art to which this disclosure relates upon reading the following description, with reference to the accompanying drawings, in which:

FIG. 1 is a schematic of an example coil device with its components in exploded form;

FIG. 2 is a schematic of a cross section of the example coil device;

FIG. 3 is a schematic of the example coil device with a flexible loop shown transparent to show the internal wire coil;

FIG. 4 is a drawing of the example coil device;

FIG. 5 is a drawing of the example coil device placed on an animal to be imaged;

FIG. 6 is one view of a drawing of the example coil device placed on a simulated rigid portion of an animal showing the flexible loop flexing;

FIG. 7 is another view of a drawing of the example coil device placed on the simulated rigid portion of an animal showing the flexible loop flexing; and

FIG. 8 shows the example coil device with added processing capability.

### DETAILED DESCRIPTION OF THE EXAMPLE EMBODIMENTS

Provided is an example embodiment of an imaging coil device for supporting the imaging of a subject (human or animal) including a small surface coil device that incorporates a flexible loop for acting as a receiving coil. The device is built so that the coil electronics, alternatively including in any combination a tune capacitor, a match capacitor, a circuit to actively decouple the coil, an in-line preamplifier, and a cable wave trap are placed in a rigid coil part, whereas the coil loop, which picks up the signal, such as a Nuclear magnetic resonance (NMR) signal, is in a flexible part. This has the advantage that the coil shape can be adapted to the imaged animal by the flexing of the flexible loop, instead of the other way around. The variable positioning and flexible loop provides a better coverage of the ROI (e.g., a sub-cutaneous tumour or the knee joint) and has hence a better filling factor and so provides an improved image SNR. The gain strongly depends on the shape of the object and the ROI but a 10% increase in the filling factor is achieved with many applications. The thin housing with its flexible shape will also allow an easier positioning when imaging regions with a rather complex shape e.g. joints, extremities or extruding malignant structures).

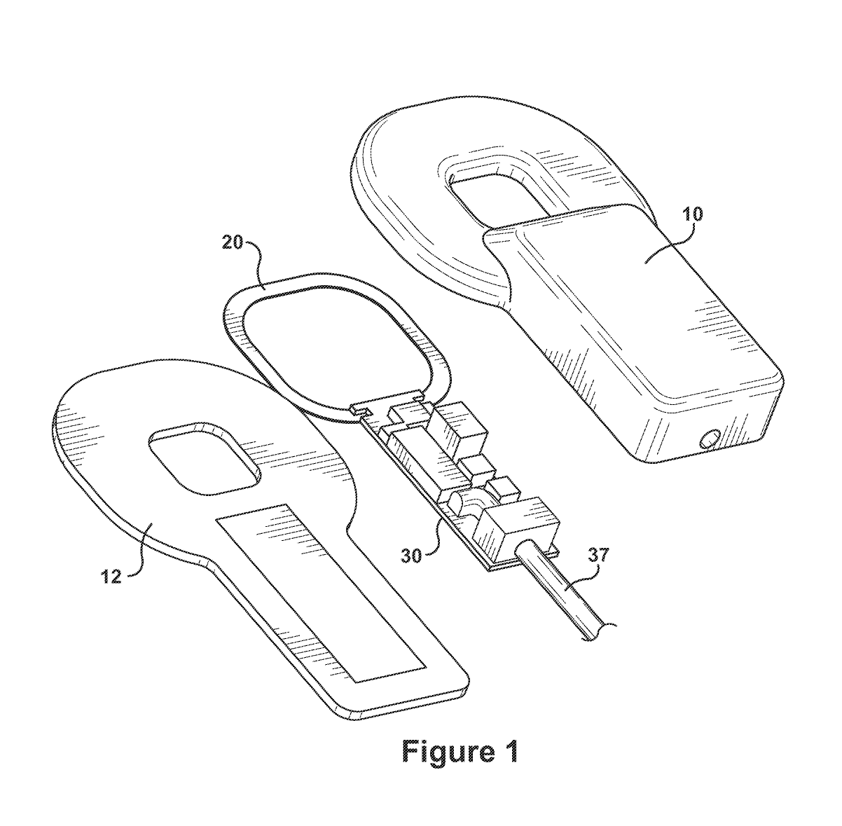

FIG. 1 shows a drawing of one example of realizing such a device for use with animals, having a flexible small animal coil. The coil electronics board 30 is comprised of a rigid part (shown as green PCB) for mounting the coil electronics including, for example, a tuning capacitor, a match capacitor, a circuit to actively decouple the coil, and a cable wave trap. A wire loop 20 comprising a loop of wires is physically and electrically attached to the coil electronics board 30. The coil electronics are covered by a flexible foam housing comprising a cover 10 and a bottom 12 which are connected (e.g., glued) together once the coil electronics is provided therein. The device has a connecting cable 37 for connecting the coil electronics to an imaging capture and analysis system, such as a computer (not shown).

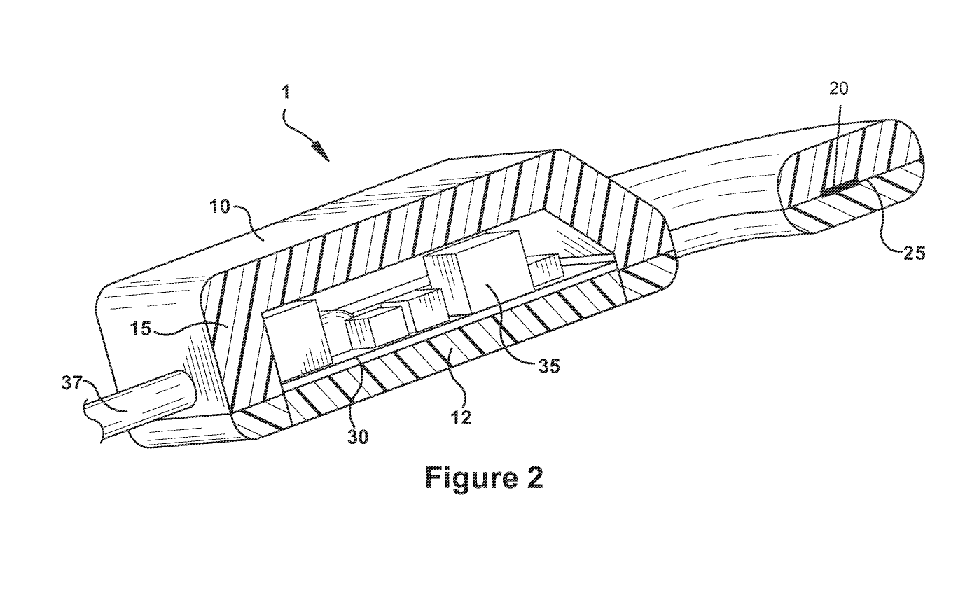

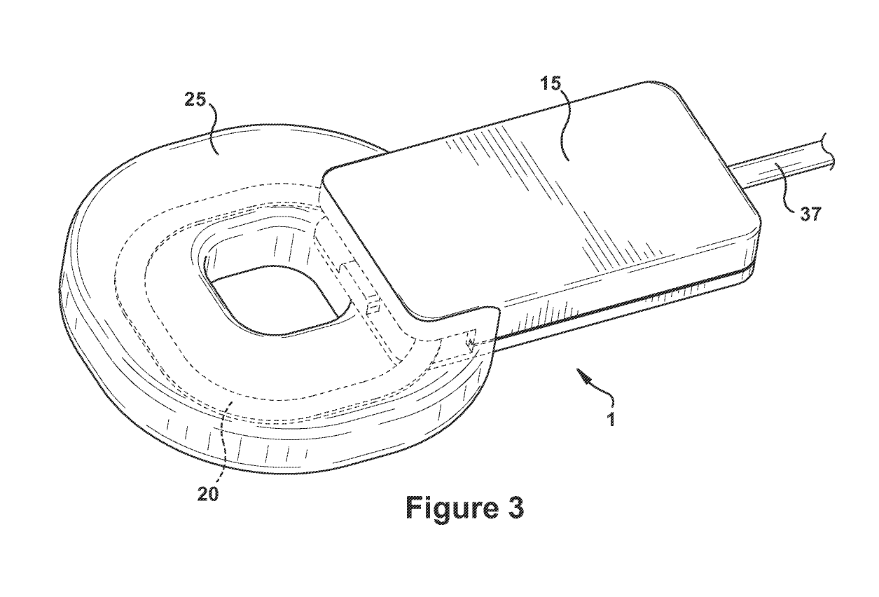

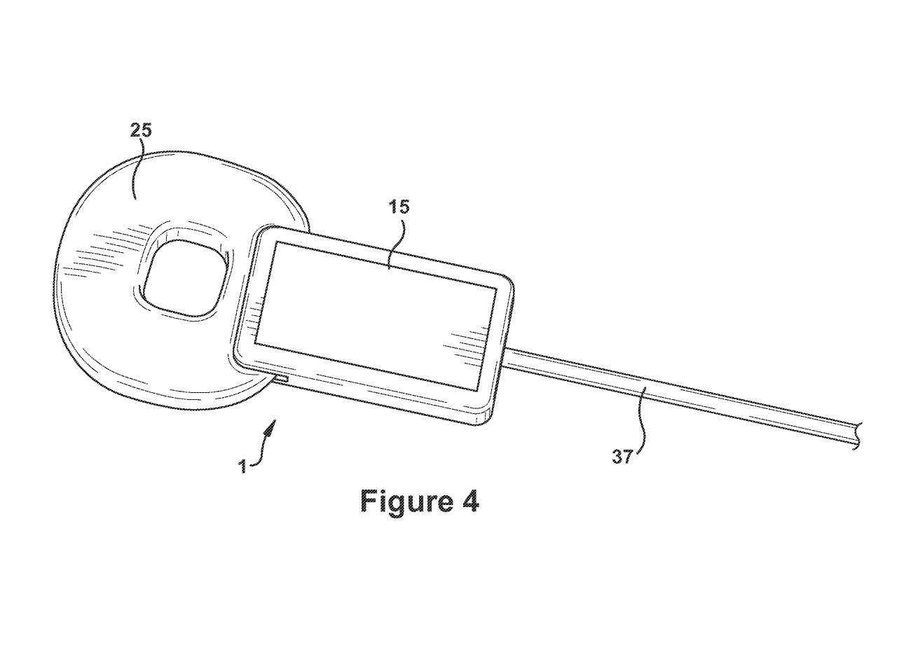

FIG. 2 shows a cross section of the assembled example coil device 1, with the cover 10 and bottom 12 glued together for housing the coil electronics board 30 with the coil electronics 35 mounted thereon. The coil device 1 has a base portion 15 which houses the coil electronics 35 and a flexible loop 25 which houses the wire loop 20. Connecting cable 37 exits the base portion 15. FIG. 3 shows the assembled coil device 1 with the base portion 15 and connecting cable 37, with the flexible loop 25 shown transparent to show the wire coil 20 housed therein. FIG. 4 shows the assembled coil device 1 with the base portion 15 and connecting cable 37, with the flexible loop 25.

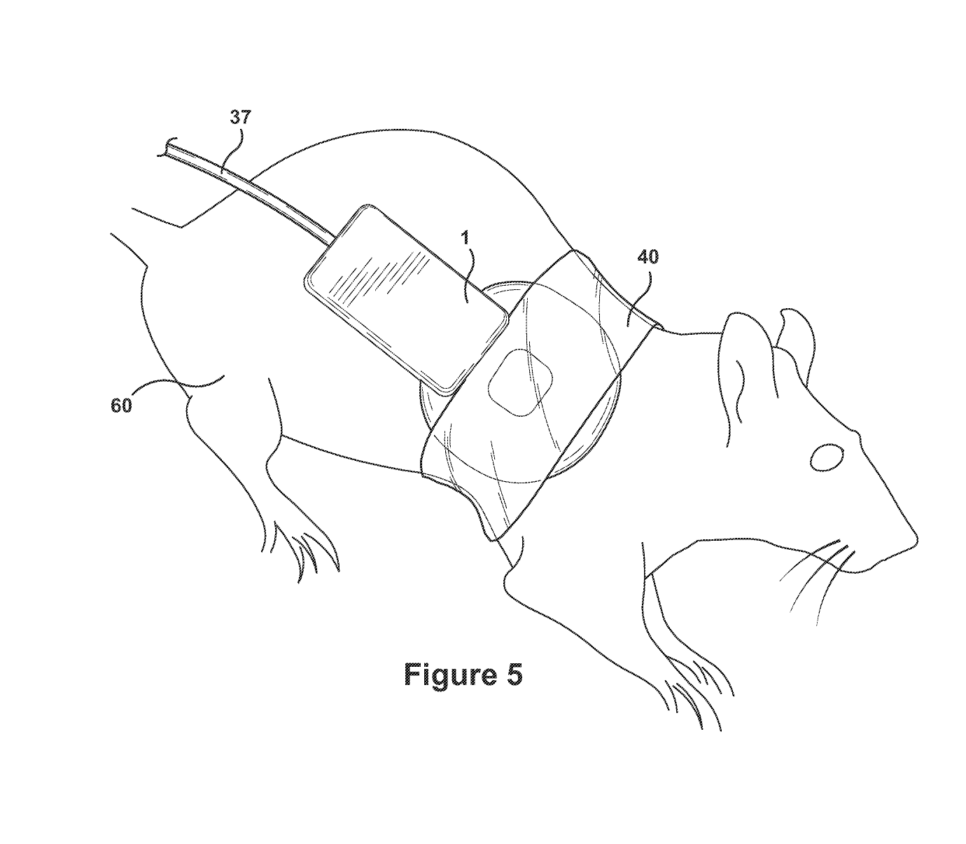

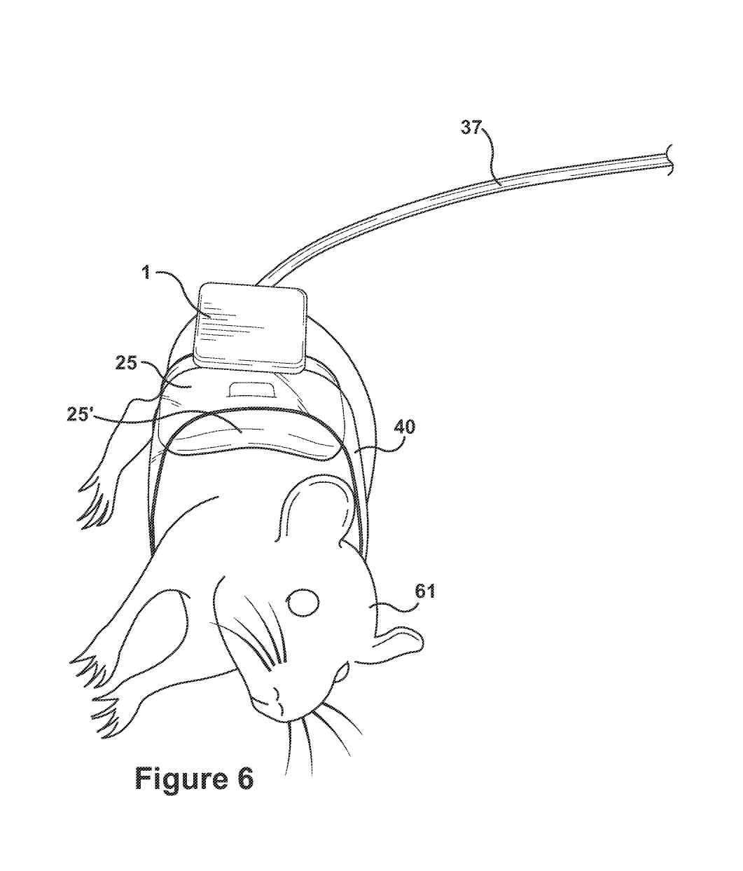

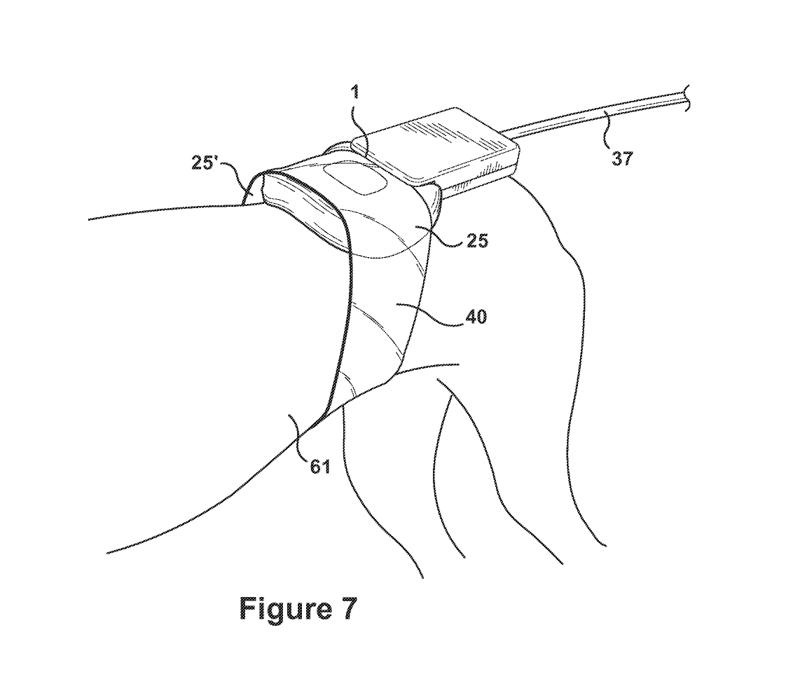

FIG. 5 shows the example coil device 1 mounted on an animal 60 to be imaged and for connecting to an external processor by cable 37. The coil is held in place with a mounting band 40. FIGS. 6 and 7 show the example coil device 1 with cable 37 mounted on a simulated animal 61 showing how the flexible loop 25 can flex around the animal 61 at point 25′

As discussed above and shown in FIGS. 1-7, the coil device 1 is comprised of a rigid coil electronics board 30 (which could also be made from several separate smaller boards on a larger flexible board) that incorporates the coil electronics 35 and protects the electronics from being torn or bent.

Attached to this board 30 is the wire loop 20 which picks up the NMR signal. The wire loop 20 is built from a cable, a flexible wire, a looped wire, or a flexible PCB material that allow a certain degree of bending to be able to adjust the coil's shape to the imaged object. The amount of bending will depend on the exercised force. There is not necessarily a limit for the bending, although in some cases for some embodiments too much force could cause the coil to break. Thus, where maximum bending is desired, this should be taken into account in the materials chosen for the wire loop 20. The bending requirements depend on the needs of the user and the type of printed circuit board (pcb). This assembly is then covered by a flexible foam-housing including a top 10 and a bottom 12. Here, the thickness of the housing can be adjusted to allow for more or less bending of the loop. With the example of the flexible small animal coil, a PE-foam (PE PF549) is used with a layer of 1.8 mm at the bottom and 4.4 mm at the top. However, with smaller coil diameters, the thickness of the housing should also be reduced.

The coil's foam housing 10,12 is constructed in a way that its bottom 12 is oriented towards the sample. For example, the housing 10,12 can be made particularly thin to minimize the distance between coil and sample and so to increase the filling factor and the image SNR. An in-line preamplifier can be provided in the electronics 35 and can be positioned after the coil to maintain the coil's SNR. This preamplifier can be built with an integrated circuit (IC) instead of single lumped elements.

To minimize standing waves on the used cable, and in order to maintain the maximum SNR, it is often advantageous to incorporate wave-traps or baluns into the coil design, such as found in Haase et. al: “NMR Probeheads for In-Vivo Applications; Concepts of Magnetic Resonance” pp 361 (2000). Incorporated herein by reference. This is used within the connection between coil and processing electronics. It a safety feature and can be incorporated into the design of coils for human use (that can then be certified as a medical product). Generally these devices supress the common mode (CM) of the signal in the cable. They are used to separate the cable into sections so that no CM standing waves can occur on the cable as a whole. This optimizes the coil performance and minimises the energy being radiated as an antenna.

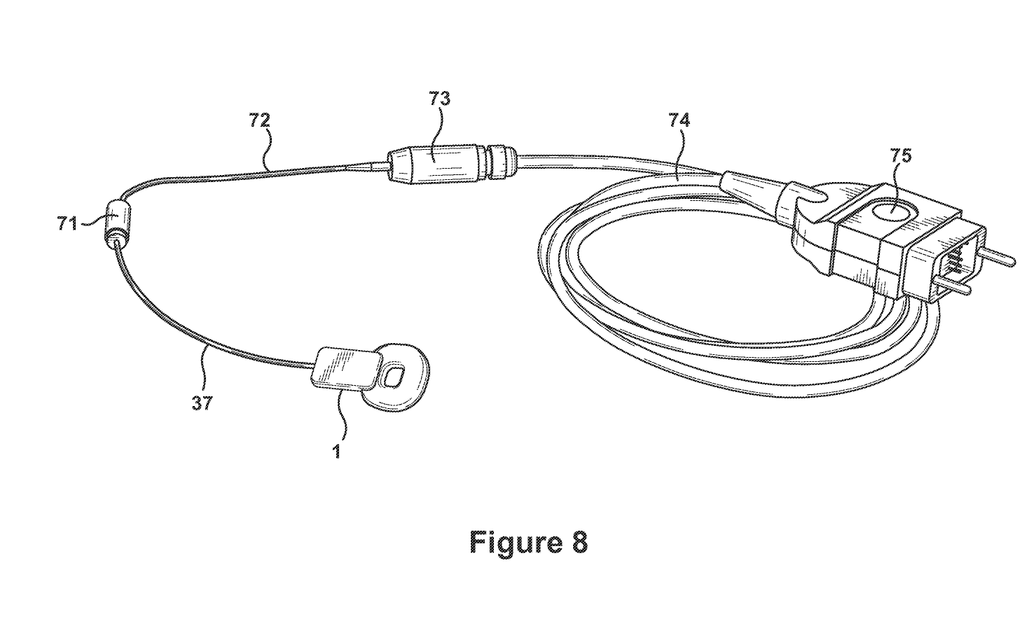

The connection between the coil device 1 and any additional components of the assembly can made with the connector cable 37. The small device can be sized as desired, including modifying the size of the flexible loop to the desired animal size, as the case may be. As shown in FIG. 8, the signal of the coil device 1 being received by the coil loop is processed by the electronics 35 and output via cable 37 through a matching network (transformation onto a 50 Ohm cable) to a wave trap 71, and then via cable 72 to a preamplifier 73 for outputting an analog signal for connecting to an external device via cable 74 (which can, e.g., provide power to the preamplifier 73) and then plug 75 for connecting to an interface of the external device to be A/D converted and Fourier-transformed in the external device (generally a computer having sufficient processing capability to support Fourier transformation) and then for further processing, storage, display, etc. Depending on the application of the coil, this data provides a spectrum that can be used for a chemical analysis or an image can be calculated. Further discussion of such processing can be found in provisional application Ser. No. 61/483,281 filed on May 6, 2011, and incorporated herein by reference.

The described device can also be doubled up where two or more devices are used together. Alternatively, a plurality of flexible loops, can be positioned in one plane and incorporated in one single flexible foam housing. Multiple flexible loops can be arranged to provide a device with a number of individual channels. As an option, such an array can be used to be operated in quadrature mode (See Haase). In such a case, a transmit quadrature arrangement can be used where two loops produce a transmit field of equal amplitude but with a phase difference of 90°. Alternatively, a receive quadrature set up could be utilized. In such a case, two individual coil channels are combined in a way that the signal from the loops is added in quadrature (with a phase difference of approximately 90°) (see Haase).

The device can be positioned easily at the region of interest (ROI) of the animal at any desired location with the use of the mounting band 40, which can be comprised of an integrated hook and loop tape or an elastic band allowing for a versatile adjustment of the coil position on the desired animal. An alternative method is to use incorporating “ears” for the option to strap the small surface coil against an animal. Such ears are loops of material connected to the device for connecting to the animal to keep it in position.

The device uses a minimum of components and structural material. It provides only a low absorption coefficient for higher energy waves (e.g. X-rays). It can be used in multi-modality imaging experiments with combinations of MR with Positron Emission Tomography (PET), Computed Tomography (CT), Single Photon Emission Computed Tomography (SPECT) or others.

The invention has been described hereinabove using specific examples and example embodiments; however, it will be understood by those skilled in the art that various alternatives may be used and equivalents may be substituted for elements and/or steps described herein, without deviating from the scope of the invention. Modifications may be necessary to adapt the invention to a particular situation or to particular needs without departing from the scope of the invention. It is intended that the invention not be limited to the particular implementations and embodiments described herein, but that the claims be given their broadest interpretation to cover all embodiments, literal or equivalent, disclosed or not, covered thereby.

What is claimed is:

1. A device for receiving an imaging signal, comprising;
a coil loop for receiving the imaging signal;
processing electronics for processing a signal received by the coil loop;
a base portion for enclosing the processing electronics;
a flexible portion for enclosing the receiving coil, wherein said flexible portion is adapted for conforming to any one of a number of different locations on the body of a subject for receiving the imaging signal from a desired region of interest of a subject being imaged.

2. The device of claim 1, wherein said flexible portion is formed into a loop.

3. The device of claim 1, wherein said processing electronics includes at least one of a tune capacitor, a match capacitor, a circuit to actively decouple the coil, an in-line preamplifier, or a cable wave trap,

4. The device of claim 1, wherein said processing electronics are provided on a rigid part to prevent said processing electronics from being damaged as said flexible portion is being conformed.

5. The device of claim 1, wherein said flexible portion and said base portion are formed from a common top portion and a common bottom portion connected together for holding said processing electronics and said receiving coil.

6. The device of claim 5, wherein said processing electronics are provided on a rigid part to prevent said processing electronics from being damaged as said flexible portion is being conformed.

7. The device of claim 1, wherein said imaging signal is one of an NMR signal, an MR signal, a PET signal, a CT signal, or a SPECT signal.

8. The device of claim 1, wherein said flexible portion is formed from a flexible foam compound having a thickness of the foam material that is adjusted to allow a desired degree of bending of the flexible coil loop to avoid damaging the processing electronics.

9. The device of claim 1, further comprising an additional receiving coil adapted for placing at a region of interest on the subject.

10. The device of claim 9, wherein two coil loops operated in quadrature mode as MR transmit coils wherein the receiving coils are arranged to each produce a transmit field of equal amplitude but with a phase difference of 90°.

11. The device of claim 1, wherein said device is used for integrating.

12. The device of claim 1, further comprising a strap for holding said device on the subject.

13. The device of claim 1, wherein said strap is a hook and loop tape to position the coil on the animal allowing for a versatile adjustment of the coil position.

14. The device of claim 1, further comprising “ears” for the option strapping the coil against the subject.

15. The device of claim 1, further comprising a wave trap or balun.

16. The device of claim 1, wherein said flexible portion with said receiving coil is adapted to be removable, and wherein said device is further adapted to receive another flexible portion with another receiving coil of a different design, shape, and/or size.

17. The device of claim 1, adapted to be used in a Multi-Modality mode in combination of NMR and PET, MR and CT, MR and SPECT, or another combination of NMR, PET, MR, CT, and SPECT.

18. The device of claim 1, wherein said processing electronics include a matching network for receiving the signal for providing onto a 50 Ohm cable, and wherein said device further comprises a wave trap for connecting to the 50 Ohm cable; and a preamplifier connected to the wave trap for outputting an analog signal to an external device for further processing.

19. A device for receiving an imaging signal, comprising;
a receiving coil for receiving the imaging signal;
processing electronics for processing a signal received by the receiving coil;
a rigid base for mounting said processing electronics thereon;
a base portion for enclosing the processing electronics and rigid base; and
a flexible portion for enclosing the receiving coil, wherein said flexible portion is adapted for conforming to any one of a number of different locations on the body of an animal for receiving the imaging signal from a desired region of interest of the animal.

20. The device of claim 19, wherein said flexible portion with said receiving coil is adapted to be removable, and wherein said device is further adapted to receive another flexible portion with another receiving coil of a different design, shape, and/or size.

21. A device for receiving an imaging signal, comprising;
a receiving coil for receiving the imaging signal;
processing electronics for processing a signal received by the receiving coil;
a rigid base for mounting said processing electronics thereon;
an individually formed top part made of a flexible material; and
an individually formed bottom part made of a flexible material, wherein
said top part and said bottom part are arranged and assembled together in a manner forming a base portion for enclosing the processing electronics and the rigid base, and wherein
said top part and said bottom part are also arranged together in a manner to form a flexible portion for enclosing the receiving coil, and further wherein
said flexible portion is adapted for conforming to any one of a number of different locations on the body of an animal held in place for receiving a signal from a desired region of interest of the animal.

22. The device of claim 21, further comprising a preamplifier for amplifying the signal received from the processing electronics for input into an external device for further processing.
